(19) United States
(12) Patent Application Publication
(10) Pub. No.: US 2013/0245501 A1
(43) Pub. Date: Sep. 19, 2013

(54) ACCURATE DILATION READER

(71) Applicant: VALERIE L. TANIS, Laurelton, NY (US)

(72) Inventor: VALERIE L. TANIS, Laurelton, NY (US)

(21) Appl. No.: 13/801,555

(22) Filed: Mar. 13, 2013

Related U.S. Application Data
(60) Provisional application No. 61/610,039, filed on Mar. 13, 2012.

(51) Int. Cl. A61B 5/00

(52) U.S. Cl. CPC A61B 5/435
USPC 600/588

(57) ABSTRACT

Apparatus that provides accurate readings and measurements of the dilation of an expectant mother's cervix during pregnancy—during and/or before the onset of labor. The apparatus envisioned by the present invention includes a speculum, control panel, prongs used to make contact with the birth canal walls, a tensile device which may be used to calculate the cervical dilation, a video camera and light source for viewing within the birth canal. The accuracy of measurements obtained by the apparatus properly indicate the expectant mother's progression of labor. Monitors may also be used in conjunction with the apparatus for viewing and measuring the cervical dilation. Additionally, processors may also be used to assist in further analysis and cervical dilation measurement.

### CROSS-REFERENCE TO RELATED APPLICATIONS

This application claims the benefit of, and is related to, provisional patent application Ser. No. 61/610,039 filed Mar. 13, 2012 and entitled ACCURATE DILATION READ which is incorporated herein in its entirety.

### FIELD OF THE INVENTION

The present invention is generally related to apparatuses directed to the measurement of the cervical dilation of an expectant mother during and/or before the onset of labor.

### BACKGROUND OF THE INVENTION

During pregnancy, the cervix of an expectant mother lengthens and thickens to protect the baby. During labor, the cervix shortens and softens to allow the baby to pass through the cervical opening. Dilation refers to the size of the cervical opening which, when measured at zero, indicates that the cervical opening is closed. Cervical dilation measured in the range of 4 to 8 cm indicates that the cervical opening is partially open and the mother is in active labor while one measured in the range of 8 to 10 cm is considered “transition” wherein the baby is being delivered. A measurement of 10 cm means the cervical opening is fully dilated or opened.

Currently, an expectant mother's cervix is measured, during the final stages of labor, by hand. A health care professional/provider inserts their hand into the woman's vagina or birth canal to approximate her dilation. Because each person's hand size is different, an approximation based on this method would vary from one person to another and may be inaccurate. Inaccurate readings of cervical thinning and/or dilation may lead to fetal distress. Additional complications may ensue because of inaccurate measurements of the cervix.

As such, there is a need for a device and/or apparatus that would provide an accurate reading of cervical dilation. There is also a need for a dilation reader device and/or apparatus that would provide uniform readings of cervical dilation.

### SUMMARY OF THE INVENTION

In light of the foregoing problem(s) it is an object of the present invention to disclose a device that would provide accurate readings/measurements of the dilation of an expectant mother's cervix during pregnancy—during and/or before the onset of labor. The device/apparatus envisioned in the present invention is meant to be used to accurately measure the size of the woman's cervical dilation. The device/apparatus, according to an aspect of an embodiment of the present invention, shall be the same size and will expand appropriately when inserted into the expectant mother's vagina or birth canal. The accuracy of the instrument's expansion, according to an embodiment of the present invention, will provide the measurements needed to properly indicate the progression of labor.

In one aspect of an embodiment of the present invention the dilation reader or speculum may include two or more prongs which may be used to gently and safely expand the vaginal walls of the expectant mother. In one aspect of an embodiment of the present invention, the prongs may retract into the dilation reader when not in use. Control of the movement of the prongs may be made possible by a control panel which an operator, such as a health care provider, may use to have the prongs expand to the vaginal wall and/or cervical opening or retract into the device once the measurements have been taken.

In one aspect of an embodiment of the present invention the dilation reader, while providing accurate readings/measurements of the dilation of an expectant mother's cervix, may also provide the health care professional/provider with a view of the cervix. In one aspect of an embodiment of the present invention, this may be accomplished by a visual component which may be located at an end of the dilation reader. In one aspect of an embodiment of the present invention, the visual device may comprise of a small video camera lens and light source.

It is yet another object of the present invention to provide a dilation reader which may be made of ergonomic tubing that would allow for its connection to any monitor where the measurements taken would be tabulated, displayed and analyzed. The body of the device, according to an embodiment of the present invention, may comprise of, or be encased in, flexible plastic tubing, which may be adapted to house the necessary wiring for control, data and power for the dilation reader's components or elements.

In one aspect of an embodiment of the present invention, the monitor may have graduations on its screen to assist in the determination of the cervical dilation. The health care professional/provider may then make a manual measurement in order to confirm the patient's diagnosis. A monitor/LCD/video screen may be used to display the view of the cervix by connecting the monitor/screen to the proximal end of the dilation reader.

In another aspect of an embodiment of the present invention, the device/apparatus may be connected with a processor/computer for analysis of the data collected and/or the measurements made. In yet another aspect, this connection may be made wireles sly.

In another aspect of an embodiment of the present invention, the prongs may be expanded to come in contact with the cervical walls in order to measure the cervical dilation. In one aspect, the tension generated within the prongs by their contact with the cervical opening may be used to determine the cervical dilation. The tension readings from the prongs and other measurements may, in one aspect of an embodiment of the present invention, be sent to a processor/computer which may then determine the size of the cervical opening or the cervical dilation.

In yet another aspect of an embodiment of the present invention, the dilation reader may include a tensile device in communication with the prongs. The tensile device may be sued, in one aspect, to determine the cervical dilation by calculating the extent to which the prongs have expanded. In another aspect, the tensile device may, with the help of a microprocessor, calculate the cervical dilation by determining the tension created within springs which work in conjunction with the prongs in their expansion and retraction.

### BRIEF DESCRIPTION OF THE DRAWINGS

The features and advantages of aspects of the present invention will become more apparent from the detailed description set forth below when taken in conjunction with the claims and drawings, in which like reference numbers indicate identical or functionally similar elements.

FIGS. 1A-1C illustrate a dilation reader with its components according to an aspect of an embodiment of the present invention.

FIG. 2 illustrates a dilation reader being used according to an aspect of an embodiment of the present invention.

FIG. 3 illustrates a dilation reader being used in conjunction with a monitor and/or computer according to an aspect of an embodiment of the present invention.

### DETAILED DESCRIPTION OF THE INVENTION

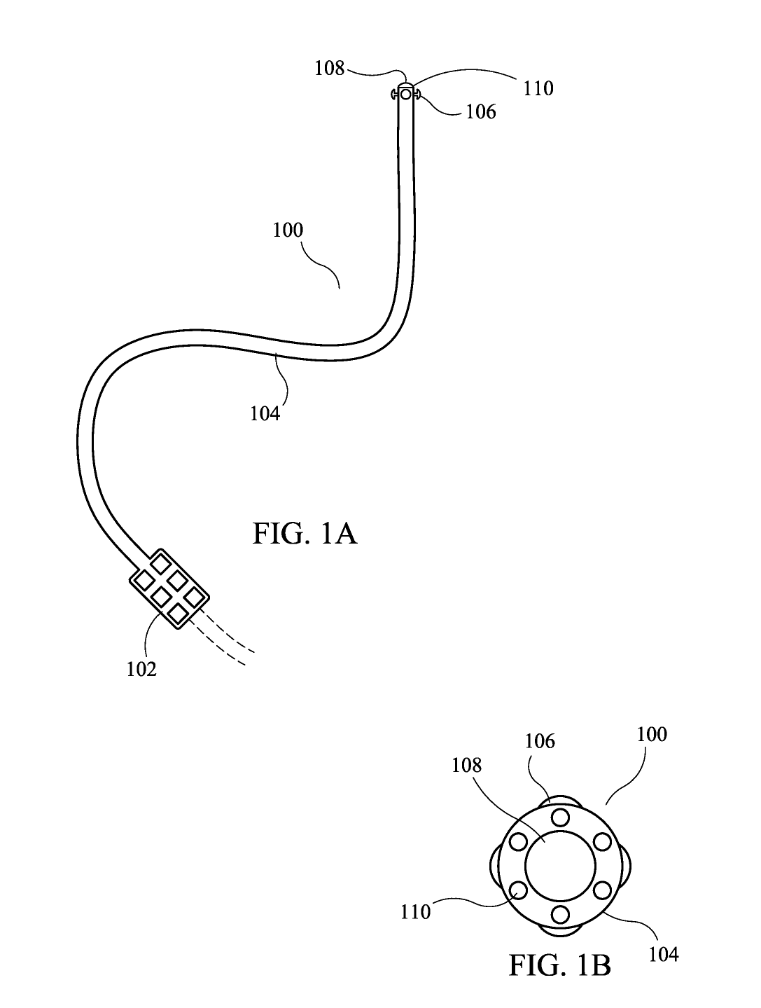

Referring now to FIGS. 1A-1C, a dilation reader 100 according to an aspect of an embodiment of the present invention is shown. In one aspect of an embodiment of the present invention, dilation reader 100 is shown having a control panel 102, ergonomic tubing 104, prongs 106, visual component 108 and light source 110. In one aspect, the control panel 102 may be used operate prongs 106—to expand or extend them beyond dilation reader 100 in order to make contact with the expectant mother's vaginal walls and/or birth canal. Control panel 102 may also be used to retract prongs 106 into dilation reader 100 once a reading has been taken or when dilation reader is not in use. Control panel 102 may additionally, in another aspect, be used to relay information obtained from dilation reader 100 to a remote device such as a monitor or a processor to analyze the readings obtained by dilation reader 100.

Visual component 108, in one aspect, may comprise of a video camera lens which, coupled with light source 110, may be used to provide an operator with a view of the vaginal walls, the cervix and/or the birth canal once the dilation reader 100 has been inserted. An operator may also be able to view the cervix with the help of visual component 108.

Ergonomic tubing 104 may, in one aspect, be made of flexible tubing to enable the device's flexibility and application. Tubing 104 may be configured to house control, data and power wiring which may be used to convey power to visual component 108 and light source 110 while also enabling control signals from control panel 102 to be relayed to prongs 106 and/or visual component 108. The video feed or picture obtained by visual component 108 may be relayed via tubing 104 to control panel 102.

In one aspect of an embodiment of the present invention, dilation reader 100 may include tensile device 112. Tensile device 112 may include sensors which determine when contact with the vaginal wall/birth canal has been made by prongs 106. The sensors of tensile device 112 in another aspect may be used to determine how wide prongs 106 have expanded away from dilation reader 100, or determine how far apart prongs 106 have expanded from dilation reader 100. In yet another aspect of an embodiment of the present invention, tensile device 112 may also include a microprocessor for determining the cervical dilation by calculation of the tension built up within springs 114 which are used to enable prongs 106 make contact with the vaginal wall and/or birth canal. This microprocessor may also be configured to calculate the dilation width by determining the tension within the springs of the device. In another aspect of an embodiment of the present invention, tensile device 112 may be connected with control panel 102 and have control signals for the operation of prongs 106 relayed through it to prongs 106. Additionally, tensile device 112 may also be linked with a remote device for relaying data obtained from prongs 106 and/or the video feed from visual component 108. Tensile device 112 may be used in conjunction with springs 114 which may be used to expand prongs 106 or retract prongs 106 after use of dilation reader 100.

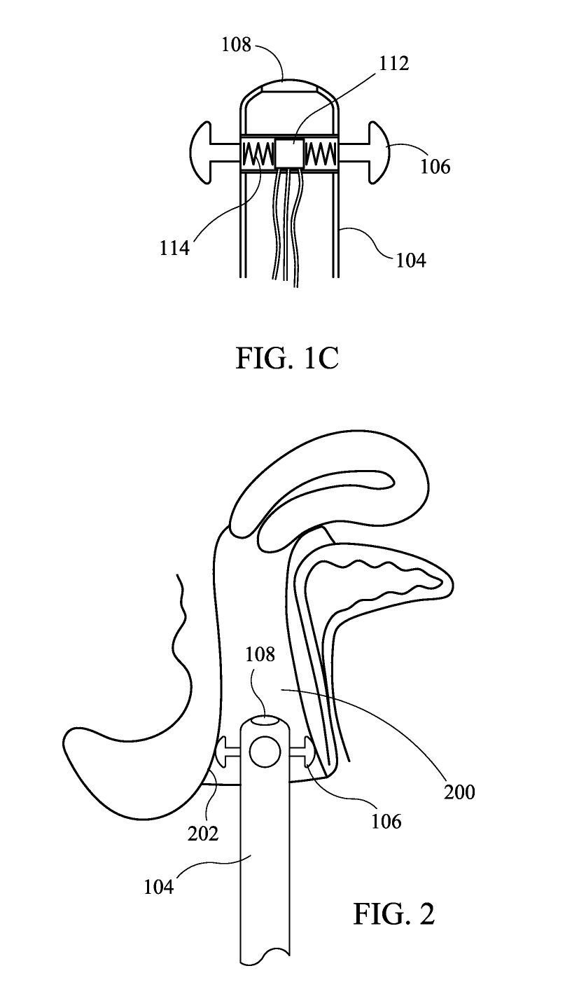

Referring now to FIG. 2, a dilation reader 100 being used within an expectant mother's birth canal 200 according to an aspect of an embodiment of the present invention is shown. When examining the cervix of an expectant mother, the health care professional/provider would first insert the device/apparatus, according to an embodiment of the present invention, into birth canal 200. Prongs 106 of dilation reader 100 would then expand out and come in contact with the vaginal walls for visual component 108 to provide an unfettered view of the cervix. With the visual component and light source 110 positioned at the end of the device, the health care professional/provider would be able to obtain an accurate visual assessment of the patient's progress with the image of the cervix that can be displayed on a monitor.

In another aspect of an embodiment of the present invention, upon positioning of dilation reader 100 within birth canal 200, an operator using control panel 102 may send a control signal via control wiring within tubing 104 to prongs 106 to expand or extend from dilation reader 100. Once contact has been made by prongs 106 with the walls of birth canal 200, tensile device 112 may then determine the cervical dilation of birth canal 200. In one aspect of an embodiment of the present invention, once prongs 106 have made contact with the uterine walls, tensile device 112 then determines the distance the prongs have moved with the help of springs 114 used in conjunction with prongs 106. In another aspect, tensile device 112 may also calculate the distance (and conversely the dilation width) by determining the tension within the prongs as a result of their contact with birth canal 200 and springs 114. The tension occurs when prongs 106 cannot expand any further once they have made contact with the walls of birth canal 200.

The data obtained from prongs 106 via, in one aspect, tensile device 112 may then be relayed via data wiring within tubing 104 to control panel 102. In another aspect of an embodiment of the present invention, a sound device may be activated once prongs 106 make contact with birth canal walls 200 and cannot expand any further at which point an operator may then take the measurements relayed to the control panel 102 and/or a monitor or processor for further display and/or analysis.

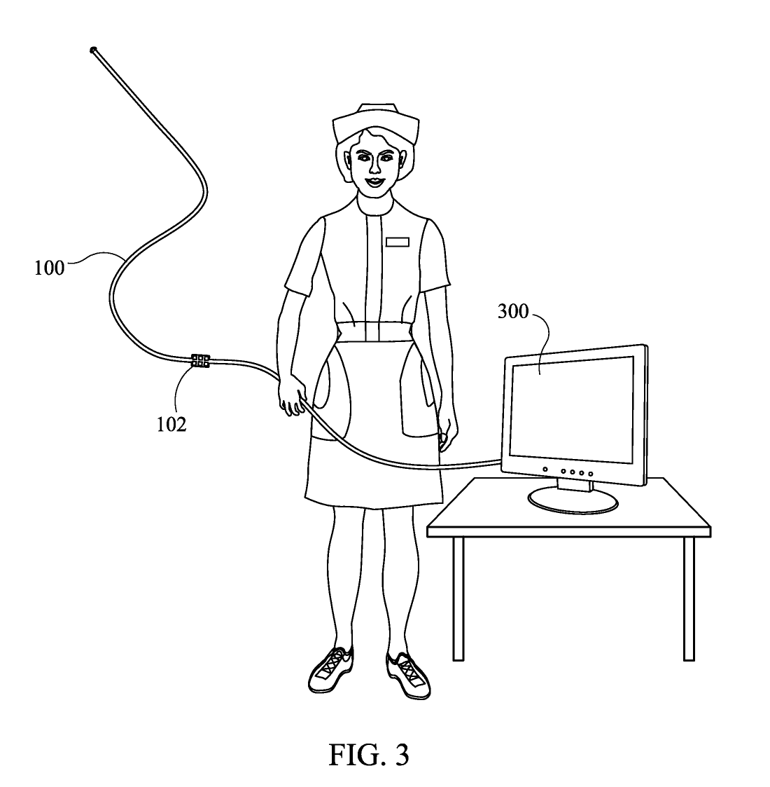

Referring now to FIG. 3, dilation reader 100 is shown as it is being used in conjunction with a monitor and/or computer 300 according to an aspect of an embodiment of the present invention. Monitor 300 may be used to display video feeds obtained from dilation reader 100 and as recorded by visual component 108. An operator may then be able to view the birth canal 200 an, in one aspect, using graduations on the monitor, determine the dilation width of birth canal 200. In another aspect of an embodiment of the present invention, monitor 300 may be connected with a processor for further analysis and measurements using the data obtained from dilation reader 100.

In another aspect of an embodiment of the present invention, the processor may be adapted or configured to calculate the cervical dilation based on the tension generated within the springs 114 once prongs 106 have made contact with the walls of birth canal 200.

Although this present invention has been disclosed with reference to specific forms and embodiments, it will be evident that a great number of variations may be made without departing from the spirit and scope of the present invention. For example, equivalent elements may be substituted for those specifically disclosed and certain features of the present invention may be used independently of other features—all without departing from the present invention as defined in the appended claims.

What is claimed is:

1. An apparatus for accurately measuring cervical dilation, comprising:
A speculum;
At least two prongs extendable from said speculum;
A tensile device within said speculum and in communication with said at least two prongs, wherein said tensile device is used to calculate said cervical dilation;
At least two springs in communication with said at least two prongs;
A video camera located at one end of said speculum; and
A light source located at one end of said speculum.

2. The apparatus according to claim 1 wherein said speculum is made of ergonomic tubing.

3. The device according to claim 1, wherein said speculum is encased within flexible plastic tubing.

4. The apparatus according to claim 3 wherein said plastic tubing is adapted to house control, power and data wiring for said speculum.

5. The apparatus according to claim 1, wherein said at least two prongs are adapted to be retractable into said speculum.

6. The apparatus according to claim 1, wherein said at least two prongs is adapted to be expandable out from said speculum.

7. The apparatus according to claim 1, wherein measurements of cervical dilation may be made by way of the tension formed within the said at least two springs in contact with said at least two prongs when said at least two prongs come in contact with the cervical walls of an expectant mother.

8. The apparatus according to claim 1, further comprising a control panel for controlling the movement and positioning of said at least two prongs in relation to said speculum.

9. The apparatus according to claim 1, wherein said speculum is connected to at least one processor.

10. The apparatus according to claim 9, wherein said processor is adapted to calculate the size of cervical dilation based on the tension within said at least two springs in contact with said at least two prongs when said at least two prongs come in contact with the cervical walls of an expectant mother.

11. The apparatus according to claim 1, wherein said speculum is connected to at least one monitor.
